(19) United States
(12) Patent Application Publication
(10) Pub. No.: US 2025/0195838 A1
(43) Pub. Date: Jun. 19, 2025

(54) FUNCTIONAL MAGNETIC ROBOT WITH LOCALIZED FLEXIBILITY

(71) Applicant: UNIVERSITY OF UTAH RESEARCH FOUNDATION, Salt Lake City, UT (US)

(72) Inventors: Yong Lin KONG, Salt Lake City, UT (US); Taylor E GREENWOOD, Salt Lake City, UT (US)

(21) Appl. No.: 18/875,152

(22) PCT Filed: Jul. 7, 2023
(86) PCT No.: PCT/US2023/027168

§ 371 Date: Dec. 15, 2024

Related U.S. Application Data
(60) Provisional application No. 63/359,648, filed on Jul. 8, 2022.

(51) Int. Cl. A61M 25/01; A61B 1/00

(52) U.S. Cl. CPC A61M 25/0127; A61B 1/00158

(57) ABSTRACT

This disclosure relates to magnetic robot devices capable of active and multidirectional transportation within a target environment such as the gastrointestinal tract. The robot device comprises a compartment configured to house one or more payload components and one or more feet joined to the compartment. At least one foot is joined to the compartment by way of a flexible connection, and the at least one foot includes a permanent magnet. The at least one foot is configured to bend relative to the compartment when exposed to a magnetic field to thereby assist in locomotion of the robot device. The compartment beneficially enables functional integration of additional components (e.g., drug and/or electronics payloads) with the robot device without disrupting effective locomotion.

### CROSS-REFERENCE TO RELATED APPLICATIONS

This Application claims priority to and the benefit of U.S. Provisional Application No. 63/359,648, filed Jul. 8, 2022 and titled “Ingestible Functional Magnetic Robot with Localized Flexibility,” the entirety of which is incorporated herein by reference.

### STATEMENT REGARDING FEDERALLY SPONSORED RESEARCH

This invention was made with government support under R21 EB029563 awarded by the National Institutes of Health, and 1830958 awarded by the National Science Foundation. The government has certain rights in the invention.

### BACKGROUND

The gastrointestinal (GI) tract plays a crucial role in the human body as a naturally-evolved interface between the body and its environment. Ingestible electronics perform surgical-free screenings and diagnoses within the GI tract and have been previously proposed. Recent advancements have demonstrated the ability to integrate ingestible electronics with sensing, actuation, and drug delivery capabilities, with several examples that have been FDA approved and are in clinical use. For example, the pill-shaped PillCam™ provides access to areas of the GI tract which are challenging or infeasible via endoscopic procedures. However, the size of an ingestible device is fundamentally constrained to enable swallowing (e.g., PillCam™ SB 3 has a diameter of 11.4 mm, and a length of 26.2 mm) and to mitigate the risks of unexpected retention (1.4% for conventional capsule endoscopes) or intestinal obstruction which requires surgical interventions. The limitation in size constrains the possible functionalities that can be integrated into an ingestible system, especially since active components such as microelectronics are rigid and planar parts that have to be assembled into the system. For example, most ingestible electronics do not have the ability to be actively transported towards target regions of interest.

Indeed, integrating functionalities into ingestible, untethered robots with active locomotion capabilities can enable a broader range of surgical-free diagnostic and treatment strategies. Earlier research has demonstrated a wide range of locomotion strategies for small-scale robots, including legged, rolling, peristaltic (i.e., earthworm-like), undulatory, crawling, and other motions. Among the demonstrated mechanisms, magnetically-controlled actuation is particularly promising because it does not require onboard power or control systems, freeing critically-needed space for additional functional integration.

Recent advances have demonstrated the ability of miniature magnetic crawlers to actively transport cargo in complex and confined systems, such as the GI tract, by leveraging magnetic fields to induce locomotion. For instance, a magnetic origami robot that crawled by in-plane contraction has previously been demonstrated. The anisotropic friction on such robot's feet enables forward locomotion that can be steered. Nevertheless, the need for anisotropic friction on the feet also precludes bidirectional locomotion in a confined space, such as in a lumen, where reversing direction by turning in place is challenging. Other recent work has demonstrated entirely-soft crawlers with multi-gait bending locomotion that could transport objects by gripping and direct attachment. Nevertheless, integrating the existing crawlers with modular electronics is challenging due to the planar and rigid nature of electronics that will impede the robot's bending motions.

Other recent work has demonstrated axisymmetric crawler robots with flexible bodies and magnetic feet. Such robots are capable of bidirectional undulatory or inchworm-like locomotion in confined lumens when actuated by an external rotating magnetic dipole. The nonuniform fields of the actuation mechanism could facilitate clinical use, as utilizing a rotating permanent magnet eliminates the need to surround the patient with coils. Nevertheless, such crawlers lack a centralized space necessary for functional integration without disrupting the robot's locomotion.

Accordingly, there is an ongoing need for improved magnetic robots that are ingestible, capable of active and multi-directional transportation within the digestive system, and that are capable of integration with functional components such as modular electronics components and/or drug payloads. Such devices would be capable of beneficially addressing several unmet clinical needs.

### SUMMARY

Disclosed herein are magnetic robot devices that are capable of active and multidirectional transportation within a target environment. The target environment can be, for example, the gastrointestinal tract. Other intraluminal environments within the body in which the robot device may be deployed include, for example, the vascular system, airways, or the urinary/reproductive system. In other implementations, the target environment can be a pipe, tunnel, underground passage, or other environment with confined spaces where inspection and/or delivery of payloads is desirable. The disclosed devices can be particularly beneficial in environments characterized by confined, lumen-shaped spaces, though the devices may be utilized in other environments that do not necessarily form lumen-shaped paths (e.g., debris zones).

The disclosed robot devices are beneficially capable of integration with functional components such as modular electronics components and/or drug payloads. Prior magnetic robot devices have not successfully leveraged localized flexibility to create a centralized compartment that can enhance the device's functionality without detrimentally affecting its gait or increasing its form factor.

In one embodiment, the robot device comprises a compartment configured to house one or more payload components and one or more feet joined to the compartment. At least one foot is joined to the compartment by way of a flexible connection, and the at least one foot includes a permanent magnet. The at least one foot is configured to bend relative to the compartment when exposed to a magnetic field to thereby assist in locomotion of the robot device.

The compartment beneficially enables functional integration of additional components (e.g., drug and/or electronics payloads) with the robot device without disrupting effective locomotion. The compartment can additionally protect such payloads in harsh environments.

The robot device is beneficially capable of controlled bidirectional locomotion within a confined target space. The bidirectional locomotion capability of the robot device is advantageous, particularly given that turning in place may not be feasible in confined spaces such as the gastrointestinal tract. The robot device is also beneficially able to turn and/or navigate bifurcations within confined spaces.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a cross-sectional view of an example magnetic robot device with features that enable active and multidirectional transportation within a target environment;

FIG. 2 schematically shows the magnetic robot device in use within a human subject who has ingested the device;

FIG. 3 is a cross-sectional view of another embodiment of a magnetic robot device that includes a lumen through which an intraluminal and/or diagnostic device may be passed;

FIGS. 4A and 4B illustrate results of locomotion testing comparing a previous magnetic robot with distributed flexibility (MR-DF) and the presently disclosed magnetic robot with localized flexibility (MR-LF), with FIG. 4A illustrating the locomotion experimental setup and FIG. 4B showing similar locomotion of the MR-DF (top) and the MR-LF (bottom), both having a mass of 2.55 g;

FIGS. 5A and 5B show results of a foot flexion test comparing the MR-DF and the MR-LF, with FIG. 5A foot flexion angle (θ_{f}) as a function of actuator magnet orientation (θₐ) for half-robots, and FIG. 5B showing images of the half-robots at their maximum (left) and minimum (right) foot flexion;

FIG. 6 shows results of a test investigating the initial speed (average speed of the first ten steps) of the MR-DF and the MR-LF, with results showing that the average initial speed of the MR-LF was faster than the MR-DF control at every actuator magnet offset tested;

FIG. 7 shows results of a test investigating how the mass of functional components and payloads within the compartment of the MR-LF affects locomotion speed;

FIGS. 8A and 8B show results of testing investigating gait characteristics of the MR-DF and the MR-LF, with FIG. 8A showing the gait type for the first ten steps (five seconds) of each experiment trial and FIG. 8B showing images during one step of each gait;

FIGS. 9A and 9B show images from an experimental drug release setup, with FIG. 9A showing release of a dye in water within a lumen as a result of temperature of the water, and FIG. 9B showing the use of a catheter associated with the robot device to deliver the dye;

FIG. 10 shows multiple robots assembled by conjoining two MR-LFs with feet with opposite polarity; and

FIGS. 11A and 11B show the MR-LF's ability to navigate turns and bifurcations in confined channels.

### DETAILED DESCRIPTION

### 1. Magnetic Robot Device Overview

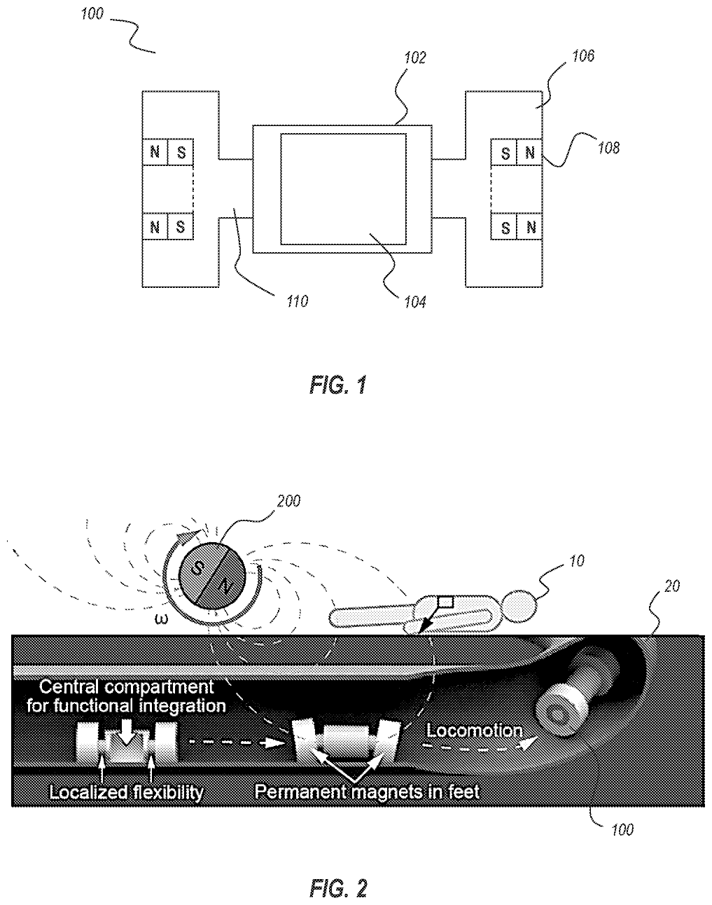

FIG. 1 is a cross sectional view of an example magnetic robot device 100 with features that enable active and multidirectional transportation within a target environment, such as within the gastrointestinal tract of a human or animal subject. The magnetic robot device 100 includes a compartment 102 that defines an inner chamber 104 in which various payload components may be placed. For example, the compartment 102 can house a drug payload and/or an electronics payload (e.g., one or more sensors) intended for delivery to a target anatomical site (e.g., to a target location within the gastrointestinal tract).

The magnetic robot device 100 also includes one or more feet 106, at least one of which is connected to the compartment 102 by way of a flexible connection 110 (also referred to herein as a “flexure”). The at least one foot 106, or in some embodiments, each foot, also includes a permanent magnet 108 integrated within the foot 106 or otherwise effectively attached thereto. In some embodiments, each foot 106 is connected to the compartment 102 by way of a flexible connection 110. Alternatively, at least one foot 106 may be connected via a flexible connection 110 while at least one other foot 106 may be connected via a rigid connection and/or may include a mechanical joint to enable pivoting movements of the foot 106 relative to the compartment 102.

Although a single foot 106 connected to the compartment 102 via a flexible connection 110 may achieve locomotion, the illustrated embodiment includes first and second feet 106 disposed on opposite ends of the compartment 102. While this opposed two-foot design has proven capable of effective multidirectional transport within a lumen environment, other embodiments may include a single foot 106 or may include more than two feet 106.

In embodiments that include first and second feet 106 aligned on opposite sides of the compartment 102, such as the illustrated embodiment, the feet 106 define the longitudinal axis of the device 100. The corresponding magnets 108 can be axially magnetized, positioned on the longitudinal axis, and arranged with alternating polarities, as shown by the “N” and “S” indicators. Although FIG. 1 illustrates the “N” poles facing outward and the “S” poles facing inward toward each other, these positions may be reversed in other embodiments.

Previous magnetic robot devices have been unable to achieve a centralized space for functional integration without disrupting the device's locomotion capabilities. The inclusion of a compartment 102 beneficially enables functional integration of additional components (e.g., drug and/or electronics payloads) with the robot device 100 without disrupting effective locomotion. The compartment 102 can also provide storage space (e.g., for tissue, fluid, and/or environmental samples acquired by the robot device 100).

The compartment 102 can beneficially be sized to carry a desired payload while being small enough to allow for ingestion. In some embodiments, the compartment 102 has an internal volume of at least about 50 mm³, or at least about 100 mm³, or at least about 150 mm³, or at least about 200 mm³, or at least about 250 mm³, or about 300 mm³, or an internal volume within a range that uses any combination of the foregoing values as endpoints. The volume of the compartment 102 is advantageously large relative to the overall size of the robot device 100. For example, the internal volume of the compartment 102 may comprise at least about 8% of the total volume of the device, or at least about 10% of the total volume of the device, or at least about 12% of the total volume of the device, or at least about 14% of the total volume of the device, or about 17% of the total volume of the device, or may comprise a percentage of the total volume of the device within a range that uses any combination of the foregoing values as endpoints.

The compartment 102 may be relatively rigid. This allows effective transport of payload components that may not be otherwise transportable. For example, certain electronics components may be rigid themselves, and thus incapable of being effectively transported using a compliant, flexible housing. Although the compartment 102 is relatively rigid, the flexible connections 110 provide sufficient flexibility to enable the feet 106 to bend relative to the compartment 102 and thereby drive locomotion of the device 100, as described in further detail below. In other embodiments, the compartment 102 may be formed from a relatively flexible material. For example, the compartment 102 may be formed from the same material as the feet 106 and/or flexible connections 110 in an integral manner.

The compartment 102 and/or the feet 106 may have a cylindrical/disk shape. As shown, the compartment 102 may have a cylindrical shape, and the flexible connections 110 may attach at end faces of the cylinder shape of the compartment 102. While the illustrated device 100 and the shapes of its components have shown to be effective, other embodiments may include a differently shaped compartment 102 and/or differently shaped feet 106. For example, the compartment 102 and/or feet 106 may have polygonal cross-sectional shapes, may have rounded edges (e.g., rather than planar end faces), and/or may comprise other suitable shapes.

The flexible connections 110 may be substantially centered with the axis of the cylinder 102, and the respective feet 106 may likewise be centered with the axis of the cylinder 102. That is, in embodiments where the feet 106 have shapes with end faces, the flexible connections 110 may attach at the center of the end face of each of their respective feet 106.

The flexible connections 110 may have lengths of about 1.25 mm to about 3.5 mm, or about 1.5 mm to about 3 mm, or about 1.75 mm to about 2.5 mm, or about 2 mm, or a length within a range that uses any combination of the foregoing values as endpoints. The flexible connections 110 may have diameters of about 2.5 mm to about 4.5 mm, or about 2.75 mm to about 4.25 mm, or about 2.75 mm to about 4 mm, or about 3 mm to about 3.75 mm, or about 3.6 mm, or a diameter within a range that uses any combination of the foregoing values as endpoints. Dimensions within such ranges were found to beneficially allow sufficient bending while preventing contact between the feet 106 and the compartment 102 (e.g., for embodiments where the feet 106 and flexible connections 110 are formed from a suitable silicone polymer).

Although the robot device 100 is not limited to use within the human gastrointestinal tract, the robot device 100 can beneficially be sized to be ingestible by a human. For example, the robot device 100 may have an overall diameter (largest outer diameter dimension as defined by the feet 106) of about 8 mm to about 15 mm, or about 9 mm to about 14 mm, or about 10 mm to about 13 mm, or about 11 mm to about 12 mm, or a diameter within a range that uses any combination of the foregoing values as endpoints, and may have an overall length of about 20 mm to about 30 mm, or about 22 mm to about 28 mm, or about 24 mm to about 26 mm, or may have an overall length a within a range that uses any combination of the foregoing values as endpoints.

The compartment 102 preferably has an outer diameter smaller than the outer diameter of the feet 106. For example, the compartment 102 may have an outer diameter that is about 40% to about 90%, or about 50% to about 80%, or about 60% to about 70% of the outer diameter of the feet 106, or the outer diameter of the compartment 102 may be a percentage of the outer diameter of the feet 106 within a range that uses any combination of the foregoing values as endpoints. A smaller compartment 102 according to the foregoing values can beneficially limit undesired contact between the feet 106 and the compartment 102 during locomotion.

FIG. 2 shows the magnetic robot device 100 in use within a human subject 10 who has ingested the device 100. A rotatable actuator magnet 200 is positioned external to the subject 10 and is rotated to generate a rotating magnetic field. The rotating magnetic field causes the feet 106 of the magnetic robot device 100 to deflect in a periodic, oscillating manner that corresponds to rotation of the actuator magnet 200, with the flexible connections 110 bend accordingly. This dynamic motion enables the robot device 100 to move in an axial direction through the target lumen 20.

Advantageously, the direction of rotation of the actuator magnet 200 corresponds to the axial direction of locomotion of the robot device 100. This enables bidirectional control over the robot device 100 by adjusting the direction of rotation of the actuator magnet 200. The bidirectional locomotion capability of the robot device 100 is beneficial, particularly given that turning in place may not be feasible in confined spaces such as the gastrointestinal tract.

The robot device 100 is also beneficially able to turn and/or navigate bifurcations within confined spaces. Turning can be achieved by adjusting the position and orientation of the actuator magnet 200 relative to the robot device 100. That is, the rotation axis of the actuator magnet 200 may be adjusted to be substantially perpendicular to the intended path of the robot device 100. Accordingly, the locomotion of the robot device 100 may be controlled by adjusting the position, orientation, and rotation direction of the actuator magnet 200.

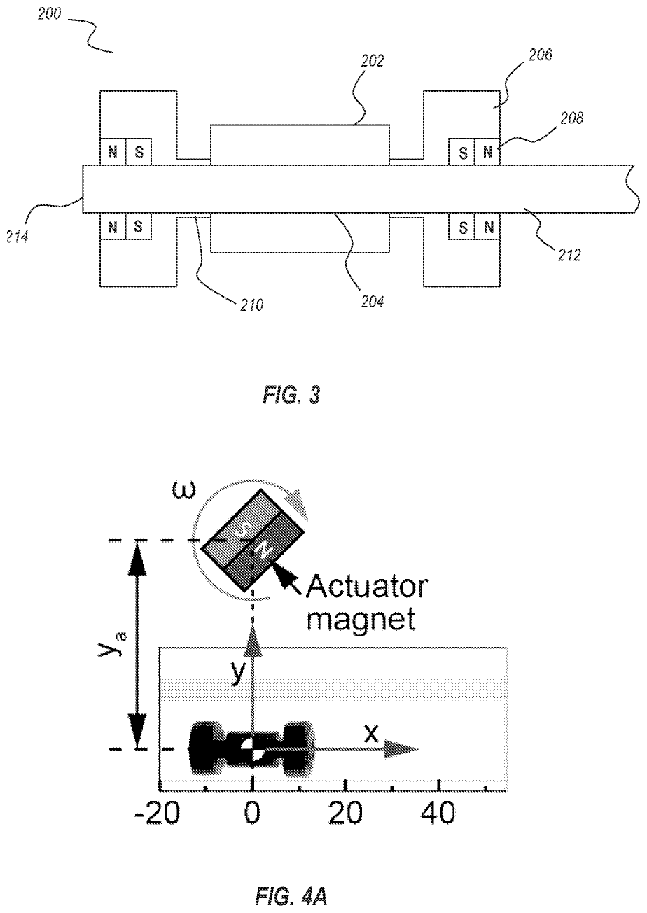

FIG. 3 is a cross-sectional view of another embodiment of a magnetic robot device 200. The robot device 200 may be similar to the robot device 100 except where specified, and like reference numbers refer to like parts in the corresponding Figures. The robot device 200 includes a center segment 202 with feet 206 and magnets 208 that connect to the center segment 202 by way of flexible connections 210. The illustrated robot device 200 also includes an internal lumen 204 that extends through the device. The lumen 204 enables the robot device 200 to be used with an intraluminal device 212. The intraluminal device 212 may be, for example, a catheter, guidewire, endoscope, or the like. Such embodiments may be used, for example, to navigate the distal end 214 of the intraluminal device 212 to targeted anatomy, where drugs, embolic fluids/devices, aspiration suction, and/or other functions can be provided by the intraluminal device.

### 2. Example Embodiments

The following clauses represent non-limiting example embodiments:

Clause 1: A robot device, comprising: a compartment configured to house one or more payload components; and one or more feet joined to the compartment, wherein at least one foot is joined to the compartment by way of a flexible connection, the at least one foot including a permanent magnet, and wherein the at least one foot is configured to bend relative to the compartment when exposed to a magnetic field to thereby assist in locomotion of the robot device.

Clause 2: The robot device of clause 1, wherein the device comprises first and second feet, each of which includes a respective permanent magnet.

Clause 3: The robot device of clause 2, wherein the first and second feet are disposed on opposite sides of the compartment.

Clause 4: The robot device of clause 2 or 3, wherein both the first and second feet are joined to the compartment by way of a respective first and second flexible connection.

Clause 5: The robot device of clause 4, wherein the first and second feet are cylindrically shaped, and wherein first and second flexible connections join at the center of the respective first and second feet.

Clause 6: The robot device of any of clauses 1-5, wherein the compartment is a cylinder, optionally wherein the robot device comprises first and second flexible connections that join to opposite end faces of the cylinder.

Clause 7: The robot device of any of clauses 1-6, wherein the flexible connection has a length of about 1.25 mm to about 3.5 mm, or about 1.5 mm to about 3 mm, or about 1.75 mm to about 2.5 mm, or about 2 mm.

Clause 8: The robot device of any of clauses 1-7, wherein the flexible connection has a diameter of about 2.5 mm to about 4.5 mm, or about 2.75 mm to about 4.25 mm, or about 2.75 mm to about 4 mm, or about 3 mm to about 3.75 mm, or about 3.6 mm.

Clause 9: The robot device any of clauses 1-8, wherein the robot device is sized to be ingestible by a human.

Clause 10: The robot device of any of clauses 1-9, wherein the compartment has an internal volume of at least about 50 mm³, or at least about 100 mm³, or at least about 150 mm³, or at least about 200 mm³, or at least about 250 mm³, or about 300 mm³.

Clause 11: The robot device of any of clauses 1-10, wherein the internal volume of the compartment comprises at least about 8% of the total volume of the robot device, or at least about 10% of the total volume of the robot device, or at least about 12% of the total volume of the robot device, or at least about 14% of the total volume of the robot device, or about 17% of the total volume of the robot device.

Clause 12: The robot device of any of clauses 1-11, further comprising a payload within the compartment.

Clause 13: The robot device of clause 12, wherein the payload comprises a drug payload and/or one or more electronic components.

Clause 14: The robot device of any of clauses 1-13, wherein the compartment is more rigid than the flexible connection.

Clause 15: The robot device of any of clauses 1-14, wherein the outer diameter of the compartment is greater than the outer diameter of the flexible connection, such as wherein the outer diameter of the compartment is greater than the outer diameter of each flexible connection.

Clause 16: The robot device of any of clauses 1-15, wherein the outer diameter of the at least one foot is greater than the outer diameter of the compartment, such as wherein the outer diameter of each foot is greater than the outer diameter of the compartment.

Clause 17: The robot device of any of clauses 1-16, further comprising a lumen extending through the robot device through which an intraluminal and/or diagnostic device may be disposed.

Clause 18: A robot system, comprising: a robot device as in any one of clauses 1-17; and a rotatable actuator magnet configured to drive rotation of the at least one foot of the robot device.

Clause 19: The robot system of clause 18, wherein adjustment of the actuator magnet's position, orientation, and rotation direction relative to the robot device enables the robot device to turn within a confined space and/or select a path at a bifurcation within a confined space.

Clause 20: A method of remotely delivering a payload to a target within a confined environment, the method comprising: providing a robot device as in any one of clauses 1-17, wherein the robot device includes the payload within the compartment; and actuating the robot device to cause locomotion of the robot device through the confined environment toward the target.

Clause 21: The method of clause 20, further comprising activating and/or releasing the payload upon reaching the target.

Clause 22: The method of clause 20 or 21, wherein the confined environment is a luminal space within human anatomy.

### 3. Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents. Similarly, while certain components may be referred to in the plural in some examples, such examples do not exclude single referents.

The embodiments disclosed herein should be understood as comprising/including disclosed components, and may therefore include additional components not specifically described.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### EXAMPLES

### 1. Body Flexibility

Abbott et al. and Leang et al. previously demonstrated soft endoluminal robots with bidirectional locomotion in lumens. See: L. N. Pham, J. A. Steiner, K. K. Leang, J. J. Abbott, IEEE Trans. Med. Robot. Bionics 2020, 2, 598; L. N. Pham, J. J. Abbott, in IEEE Int. Conf. on Intelligent Robots and Systems, IEEE, Piscataway, NJ 2018, pp. 1783-1788; and J. A. Steiner, L. N. Pham, J. J. Abbott, K. K. Leang, J. Mech. Robot. 2022, 14, 051002. However, the magnetic robot in such previous work did not have a centralized compartment for functional integration. Such a robot is referred to in this Examples section as “magnetic robot with distributed flexibility” (MR-DF) as its gait motion relies on the flexibility of its entire body. The magnetic robot device comprising a centralized compartment as disclosed herein is referred to in this Examples section as “magnetic robot with localized flexibility” (MR-LF).

We demonstrated that the MR-LF design preserves the bidirectional locomotion characteristics and ingestible form factor of the MR-DF. We modified the robot geometry to localize bending to small regions, or flexures, near each foot to convert the remaining central body length into a compartment for functional integration. The locomotion of MR-DF was shown to primarily depend on body flexibility and foot rotation induced by the magnetic field of the actuator magnet. Thus, to preserve locomotion, the MR-LF flexure geometry was designed to yield similar foot flexion angle as the MR-DF control under an applied torque. The length of the MR-LF flexure was selected as 2 mm to provide sufficient length for bending while preventing contact between the foot and compartment, and the diameter was determined to be 3.6 mm.

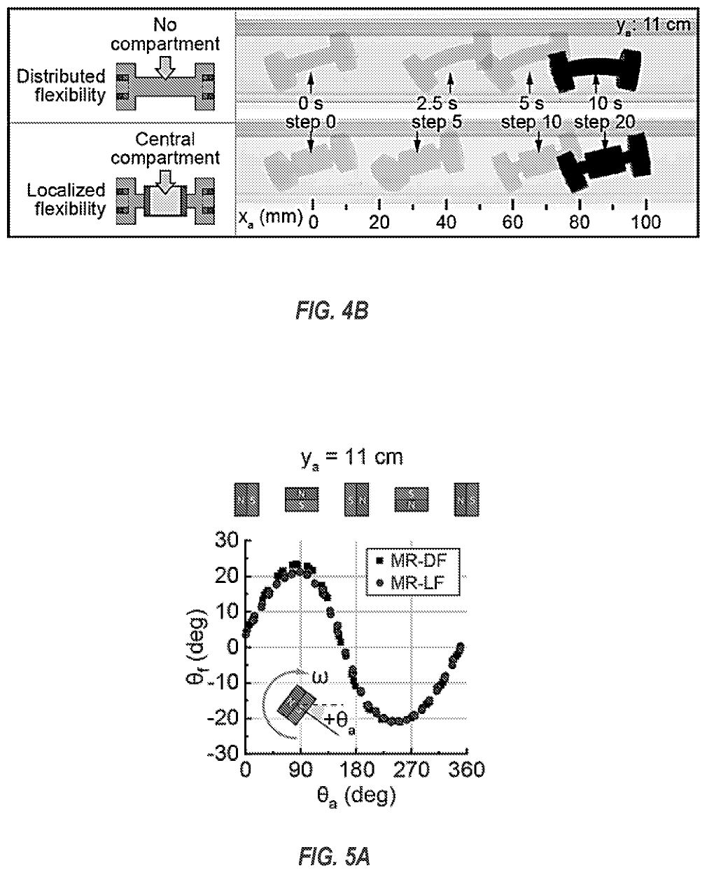

FIG. 4A illustrates a locomotion experimental setup comparing the MR-DF and MR-LF. A robot in a confined channel was actuated by a rotating actuator magnet at a distance yₐ relative to the robot's initial position (x=0, y=0). FIG. 4B shows results of the MR-DF (top) and the MR-LF (bottom), both having a mass of 2.55 g, demonstrating similar locomotion at yₐ=11 cm with time and step number labels and a displacement scale shown.

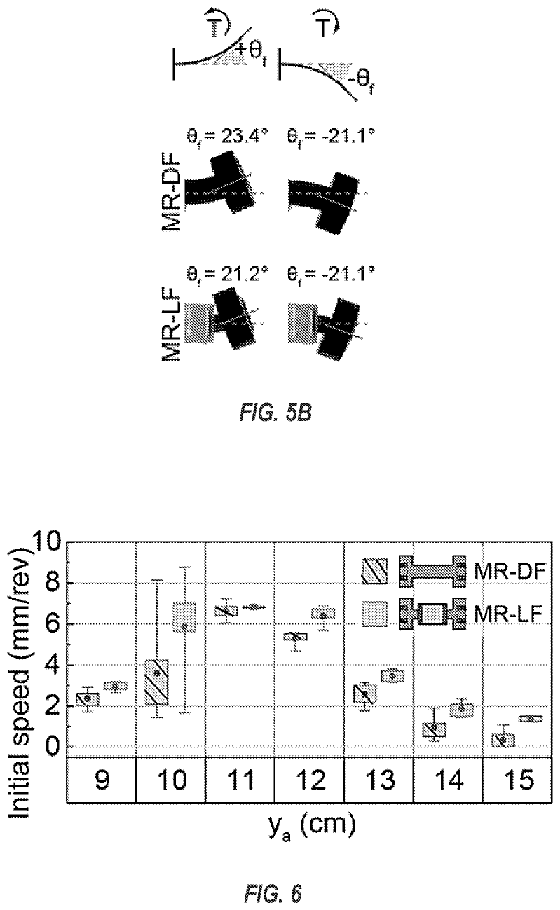

The foot flexion of the MR-LF and the MR-DF control was compared using physical half-robot models mounted at a set location (x=0, ya=11 cm) in the magnetic field of the actuator magnet. Data of the foot flexion angle across the actuator magnet orientation and images of the maximum and minimum angles are shown in FIGS. 5A and 5B. FIG. 5A shows foot flexion angle (θ_{f}) as a function of actuator magnet orientation (θₐ) for half-robots at x=0, yₐ=11 cm. The amplitude of data sets indicates the foot's flexibility. FIG. 5 B shows images of half-robots at their maximum (left) and minimum (right) foot flexion.

The minimum foot flexion of both models was the same (θf=−21.1°) , while the maximum flexion of MR-DF (23.4°) was slightly higher than MR-LF (21.2°). The MR-LF data closely match MR-DF across the entire actuator magnet rotation, which indicates that the methods to localize body flexibility were successful at reducing the bending region length while preserving foot flexion.

An upper limit exists for the body flexibility of MR-DF (which depends on body diameter, body length, and material stiffness) to prevent the robot body from buckling due to the attraction between magnets of the opposite feet. Here, by introducing a rigid segment as the compartment that prevents buckling, the MR-LF design can have a longer body, lower material stiffness, and smaller flexure diameter than MR-DF designs, which could beneficially increase robot step size and speed. Additionally, increasing the body length of the MR-LF design could enlarge the compartment and thus increase functionality.

### 2. Robot & Compartment Size

The geometry changes of the MR-LF relative to the MR-DF control did not affect the overall size of the MR-LF, as the outer diameter (12 mm) and length (25 mm) were the same as the MR-DF control. These dimensions are comparable to commercial capsule endoscopy devices such as the PILLCAM SB 3 (diameter 11.4 mm, length 26.2 mm) and PILLCAM COLON 2 (diameter 11.6 mm, length 32.3 mm), showing that the robot could be used as an ingestible device at its current scale. Further scaling down of the MR-LF is possible due to the low part count (six total parts), bending-type actuation, and commercial availability of permanent magnets with diameters lower than 1 mm. Additive manufacturing processes can also be utilized to assist in further miniaturization.

The MR-LF central compartment has an internal volume of 300 mm³ (length: 7.8 mm, diameter: 7 mm) which comprises 17% of the robot's volume (1725 mm³). In comparison, most of the prior robots with compartments have smaller compartment-to-robot volume ratios. While some previous works have demonstrated a similar order of compartment-to-robot ratio, those have been limited to the millimeter length scale that is not compatible with the goal of ingestible electronics. For example, a millimeter-scale multigait magnetic robot (see W. Hu, G. Z. Lum, M. Mastrangeli, M. Sitti, Nature 2018, 554, 81) has a compartment volume of 2.5×10⁻² mm³, which is 120,000 times smaller than the compartment in MR-LF.

Another example leverages a magnetically actuated cylindrical compartment (see Y. Wu, X. Dong, J. Kim, C. Wang, M. Sitti, Sci. Adv. 2022, 8, eabn3431) with a compartment-to-robot ratio of ˜36% but with a ˜500 times smaller volume than MR-LF. In another example, an ingestible magnetic origami crawler (see Q. Ze, S. Wu, J. Nishikawa, J. Dai, Y. Sun, S. Leanza, C. Zemelka, L. S. Novelino, G. H. Paulino, R. R. Zhao, Sci. Adv. 2022, 8, eabm7834) demonstrated a compartment for a cargo volume that is 12.5× smaller than MR-LF (24 mm³, ˜7% of robot volume) that is not centralized (3.5% on each robot end) and requires fixed-free mounting to preserve actuation. Thus, in contrast to previous works, MR-LF localized flexibility endows the device with a large (300 mm³) centralized compartment that can be integrated with functional modules (e.g., electronics) within an ingestible form factor.

### 3. Effect of Geometry on Speed

To investigate the effect of localizing flexibility on locomotion speed, experiments were performed using an MR-DF and an MR-LF with an equal mass (2.55 g). In each experiment, the robot was placed in a confined channel and actuated by a rotating actuator magnet at x=0, y=yₐ (FIG. 6). The rotation of the actuator magnet about the-z axis induced locomotion in the +x direction due to the phase shift in time-varying torque on the robot feet. A range of yₐ values was studied because existing literature reported a relationship between yₐ and locomotion. FIG. 6 shows the initial speed (average speed of the first ten steps) of the MR-DF and the MR-LF. Boxes show the 25-75% range, whiskers indicate min-max, and markers indicate mean (n=5).

Results show that the average initial speed (average speed for the first ten steps of locomotion) of MR-LF was faster than the MR-DF control at every yₐ offset. At yₐ=11 cm, the robots had the closest speeds (difference of 3%) and exhibited their fastest average initial speed (MR-DF: 6.61 mm/rev, MR-LF: 6.82 mm/rev). The largest difference (299%) and slowest average initial speed for both designs were at yₐ=15 cm (MR-DF: 0.34 mm/rev, MR-LF: 1.37 mm/rev).

As anticipated, the closeness in robot speeds is likely due to the comparable foot flexion between the designs (0% difference in minimum, 10% difference in maximum foot flexion). The superior performance of MR-LF, which had an average initial speed of 0.21 to 2.27 mm/rev faster than MR-DF across all yₐ, may be due to difference in mass distribution between the robots or the 10% reduction in maximum foot flexion. The closeness in locomotion performance between the MR-DF and MR-LF designs and the superiority of MR-LF across all yₐ is demonstrates that localizing flexibility yielded a 3 to 299% increase in speed while also freeing up space for an internal compartment (300 mm³) for functional integration.

In this experiment, the robot's locomotion was measured away from the actuator magnet to demonstrate the feasibility of locomotion against the attraction forces between the robot and actuator. In practice, robot speed and endurance can be improved by having the robot travel toward the actuator magnet and actively modulating the separation between the actuator and robot.

### 4. Effect of Robot Mass on Speed

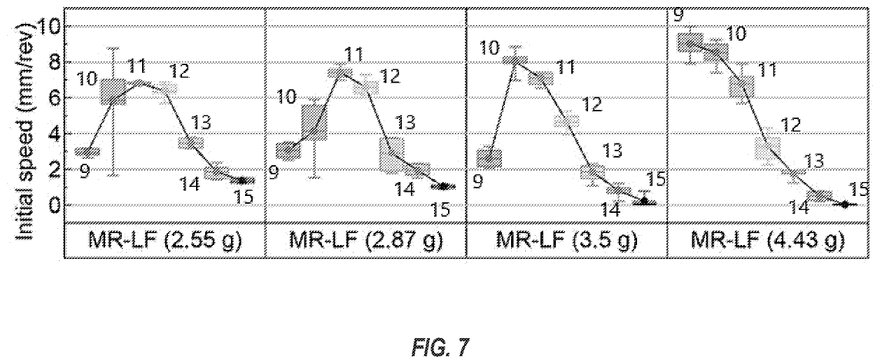

To investigate how the increased mass of functional components and payloads within the compartment affects locomotion speed, experiments were performed using MR-LF with varying mass (2.55, 2.87, 3.5, 4.43 g). Comparison between plots shows that, in general, increasing MR-LF mass increases the initial speed at smaller yₐ values (indicated by the numbers adjacent to the boxes of the plots, in cm) and lowers the initial speed at larger yₐ values within the studied yₐ range (FIG. 7). At the smallest offset (yₐ=9 cm), the heaviest MR-LF (4.43 g) exhibited the fastest average initial speed (9.01 mm/rev), while the other MR-LFs were unable to exhibit effective locomotion (discussed in the next section). Conversely, at the largest offset (yₐ=15 cm), the heaviest MR-LF (4.43 g) exhibited a low average speed (0.02 mm/rev), while the 2.55 g and 2.87 g MR-LFs had average speeds greater than 1.0 mm/rev. The difference in speeds is due to the mass of the robots, as robots with higher mass can resist a higher lift force that is generated by a higher magnetic field strength.

Our results demonstrated that MR-LF achieved locomotion even with a mass greater than existing capsule endoscopy devices (PILLCAM SB 3:3.0 g, PILLCAM COLON 2:2.9 g). Indeed, the heaviest robot in our experiments exhibited the fastest average initial speed (9.01 mm/rev), and results show that increasing robot mass can improve locomotion speed and change the yₐ for the fastest locomotion.

### 5. Gait Type

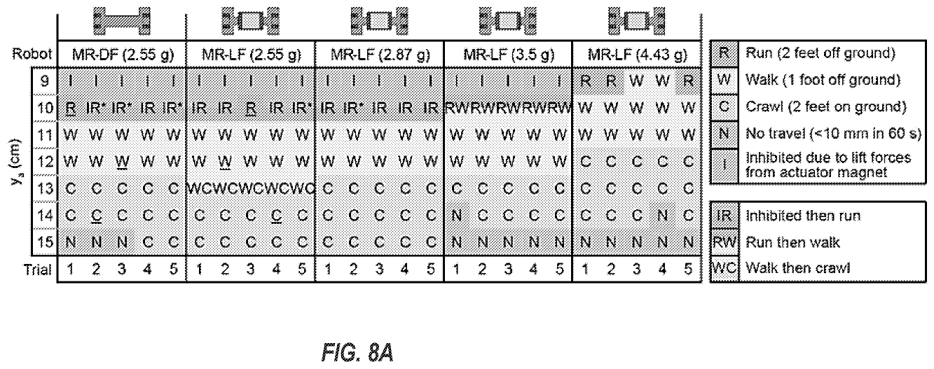

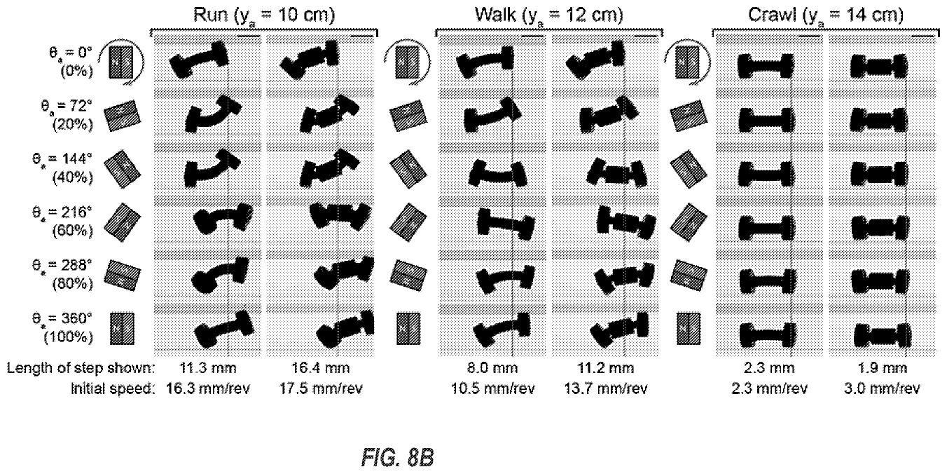

To analyze the locomotion characteristics of MR-DF and MR-LF, the gait type for the first ten steps (five seconds) of each experiment trial was identified and organized as shown in FIG. 8A. Gait was classified into five categories: run, walk, crawl, no travel, and inhibited. Images during one step of each gait are shown in FIG. 8B. In FIG. 8B, the approximate θₐ and corresponding step percentage are included on the left. The length of the displayed step and the initial speed (average speed for the first ten steps) are labeled below the image sequences. Vertical lines are aligned with the robot's leading foot at θₐ=0° to show the distance traveled during the step. Scale bars: 10 mm.

In the “run” gait, both robot feet were simultaneously off the ground at some instances during locomotion; in the “walk” gait, only one foot was off the ground at a time; and in the “crawl” gait, both feet stayed in contact with the ground. If the robot was unable to move more than 10 mm during the 60-second test and remained in contact with the bottom of the lumen, the gait is defined as “no travel.” If the robot was lifted and unable to move due to the lifting forces of the actuator magnet, the gait is defined as “inhibited.” We reported the gait type exhibited during a majority of the first ten steps as, in some cases, the differences between gait types could be subtle. We reported a combination of gait types in cases (e.g., during transition) where the robot exhibited an approximately equal ratio of two gait types.

The data show a relationship between gait type and yₐ. For instance, trials at larger yₐ (13 to 15 cm) exhibited the “crawl” gait where both feet remain on the ground, and trials at smaller yₐ (9 to 12 cm) exhibited gaits where feet are lifted off the ground (e.g., run, walk) due to the higher magnetic field strength. Moreover, as the mass of MR-LF increased, a higher magnetic field strength (i.e., smaller yₐ) was required to produce the same gait type. For instance, at yₐ=12 cm, the lightest MR-LF exhibited a “walk” gait, while the heaviest MR-LF exhibited a “crawl” gait. As noted in the previous section, the high magnetic field strength at yₐ=9 cm caused all robots except the 4.43 g MR-LF to be lifted to the top of the channel and exhibit an “inhibited” gait. Similarly, the magnetic field strength at yₐ=10 cm resulted in an “inhibited” gait during some of the experiments (“IR*” classification applied when effective run gait began after the tenth step). Due to this transition between “run” and “inhibited” gait at high field strengths, the “run” gait may not be as desirable as the “walk” or “crawl” gaits if the goal is to maintain consistent robot locomotion.

Representative images, shown in FIG. 8B, demonstrate the “run,” “walk,” and “crawl” gait types of the MR-DF and the MR-LF of equal mass (2.55 g). A comparison between the images demonstrates that localizing flexibility enabled the creation of a centralized compartment without impeding the robot's locomotion characteristics.

### 6. Functional Integration & Payload Transportation

The ability to integrate modular components and payloads can functionalize ingestible magnetic crawler robots with advanced sensing, actuation, and drug delivery capabilities that can ultimately enable a broad range of surgical-free diagnostic and treatment strategies. Here, we show that the centralized compartment in MR-LF enables the integration of modular electronic components which are otherwise challenging to integrate into soft robots due to their rigid and planar architectures. The MR-LF compartment also facilitates the incorporation of payloads such as medications for drug delivery and can potentially provide storage space for tissue and fluid samples acquired by the robot.

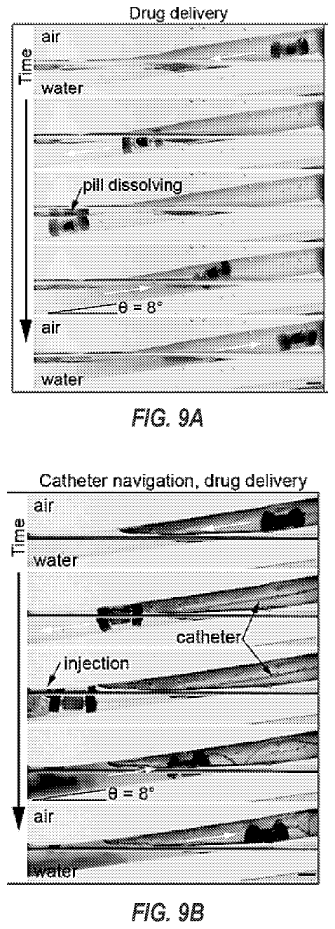

In the experiment shown in FIG. 9A, medication release (a dye dissolvable in water in this example) is triggered by the temperature of the water environment. The MR-LF rigid compartment can be entirely replaced with a soft material (“MR-LF-S”) without a loss of locomotion characteristics. The design of MR-LF-S can incorporate an internal lumen, which can enable novel treatment and diagnosis in a highly confined region. For instance, MR-LF-S can guide and transport diagnostic tools (e.g., endoscope) or enable local delivery of drugs, as shown in FIG. 9B.

These tests also demonstrated that the centralized compartment does not compromise the robot's bidirectional locomotion in water and when moving between water and air environments, even on a slope with an angle of 8°. Bidirectional motion is critically useful in a confined region where reversing direction by turning is challenging such as in the lumens of the human body.

In addition, the MR-LF design was able to overcome obstacles and push obstacles in the lateral direction, as may be necessary in confined and complex environments.

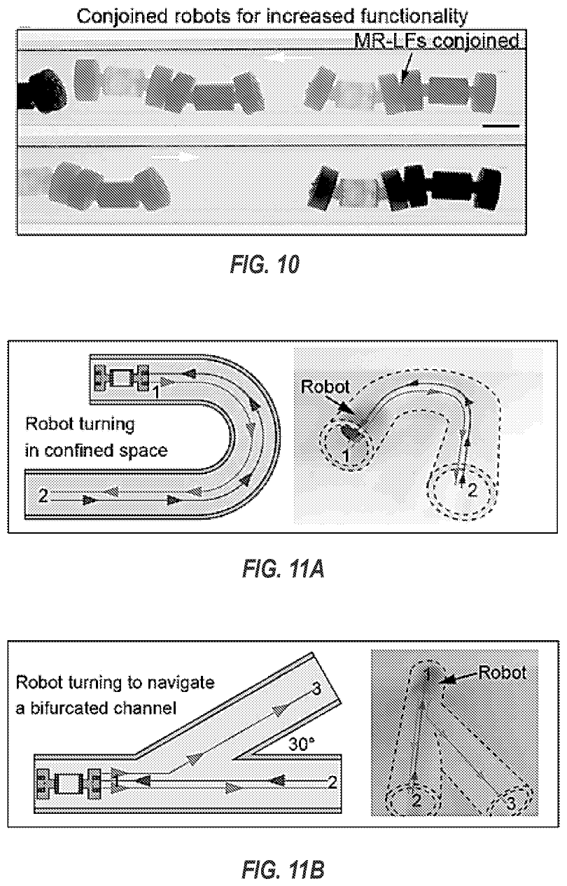

FIG. 10 shows that multiple robots can be assembled by conjoining two MR-LFs with feet with opposite polarity. Such capability shows that the functionality is not limited to a single dosage form, as multiple drug payloads can be taken to further increase functionality and payload.

We also demonstrated MR-LF's ability to navigate turns and bifurcations in confined channels (FIG. 11A and 11B). The turning was achieved by controlling the position and orientation of the actuator magnet in the x-z plane at yₐ=11 cm such that the rotation axis was approximately perpendicular to the robot's path.

The MR-LF is beneficially capable of bidirectional locomotion in a confined lumen where reversing direction by turning in place is challenging. As shown in FIG. 11A, the robot started at location 1 and moved forward around the 180° bend. At location 2, the actuator magnet rotation direction was reversed such that the robot reversed direction and moved backward around the bend to location 1.

Similarly, in FIG. 11B, the robot moved forward from location 1 to 2, backward to location 1, then forward through the other side of the bifurcation to location 3. The turning and bidirectional locomotion capabilities can be combined to enhance the navigation ability of MR-LF. More complicated robot trajectories could be achieved by controlling the actuator magnet's position, orientation, and rotation direction.

### 7. Experimental Details

a. Locomotion Experiments

In each locomotion experiment, the robot was placed in a clear polycarbonate circular channel (inner diameter [ID]: 19 mm) with the robot center at x=0, y=0. For locomotion and bending experiments, the cylindrical actuator magnet (DY0X0-N52, K&J Magnetics) was located at a fixed position (x=0, y=yₐ) with the south pole initially pointing in the +x direction. Next, the actuator magnet was rotated by a geared DC motor at a fixed voltage, producing a frequency of 2.0±0.1 Hz with rotation about the −z axis. The direction of robot locomotion is oriented away from the fixed location of the actuator magnet (i.e., away from x=0) to demonstrate the feasibility of moving against the attraction forces between the robot and actuator. Five trials were performed for each robot across yₐ offsets from 9 to 15 cm. Displacement was measured by tracking the center of the robot from a video recording of the test (Canon EOS 80D, frame rate: 29.97 fps). Displacement was calculated every 15 frames or approximately one measurement per step. The initial speed was calculated by dividing the total displacement in the first ten steps by the number of steps. For variable-mass experiments, weight was added inside the rigid compartment of MR-LF. Variability in locomotion test results could be due to several factors, including slight variations in robot starting position and magnet rotation frequency and minor fabrication defects. To reduce potential data bias from possible imperfections in the robot's feet, the channel and robot were rotated about the x-axis between trials so different portions of the foot were in contact with the floor.

b. Robot Fabrication

Robots were cast in 3D-printed molds (Form3, Formlabs) from addition-cure silicone (Dragon Skin™ 10 Medium, Smooth-On) with pigment (Silc Pig™, Smooth-On) added to aid visualization. Separate molds were used for the MR-DF, MR-LF, and MR-LF-S designs. Before casting, the molds were coated with a release agent. The silicone was mixed at a 1:1 ratio (w/w, parts A: B) in a planetary centrifugal mixer (AR-100, Thinky) for 60 seconds at 2000 rpm, then injected into the molds using a syringe and dispensing tip. After casting, magnets (R422-N52, K&J Magnetics) were glued into cavities in each foot using a silicone adhesive (Sil-Poxy™, Smooth-On) and cured overnight. Magnets were aligned coaxially with opposite polarity (i.e., both north poles pointing out). The two-part rigid compartment in MR-LF was 3D printed (Form3, Formlabs), assembled, and placed into the MR-LF molds before casting. The flexure was joined to the compartment by having silicone from the flexure extend into a cavity at the end of the compartment during casting. The overall length (25 mm), foot diameter (12 mm), and foot length (5 mm) were the same for all robots. The body length and diameter of MR-DF were 15 mm and 5 mm, respectively. The body geometry of MR-LF was as follows: flexible segment length 2 mm, flexible segment diameter 3.6 mm, compartment length 11 mm, and compartment diameter 8 mm. The compartment's outer diameter was designed to be small enough to prevent undesired contact between the robot body and channel. The MR-LF-S had the same geometry as MR-LF and was fabricated with the methods described above, except the feet, flexures, and compartment were cast as a single unit from the silicone. Half-robot models for body flexibility tests were fabricated using the same methods as described above using half-robot molds. The single magnet was glued (with the north pole out) into the foot, and the midsection face was glued to a custom 3D-printed PLA mount using the silicone adhesive and cured overnight. All full-robot models were fabricated with the same batch of silicone to avoid variation due to material properties. Similarly, all half-robot models were made from the same silicone mixture.

c. Body Flexibility Calculations & Experiments

To localize body flexibility, calculations were performed to determine the MR-LF geometry that would yield the same foot flexion as the MR-DF design while limiting the bending in MR-LF to a small region, or flexure, near each foot. The length of the MR-LF flexure was chosen as 2 mm to avoid contact between the foot and compartment, and the diameter was determined using cantilever beam equations. The robots were modeled as a cantilever beam with the midsection fixed and a constant torque applied to the free end, with the assumption that bending was symmetric on both sides of the robot and resulted from a uniform torque applied on the robot foot. For the MR-DF design, bending was assumed to occur across the entire half-body length (7.5 mm), whereas, for the MR-LF design, the compartment was assumed to be rigid so bending only occurred in the MR-LF flexure (length: 2 mm). By setting the maximum bending angle of each case to be equal, the diameter of the MR-LF flexure was calculated to be 3.6 mm.

To evaluate the effect of localized flexibility on the overall body flexibility, experiments were performed using physical half-robot models mounted with the midsection face at x=0 in the magnetic field created by the actuator magnet (FIGS. 5A and 5B). Body flexibility was determined using the maximum and minimum foot flexion angles for the designs. Foot flexion was measured from a video of the half-robot models being actuated through three rotations of the actuator magnet. The θₐ data were adjusted to represent θₐ during a representative rotation of the actuator magnet.

d. Functional Integration & Payload Transportation Experiments

To demonstrate functional integration and payload transportation enabled by MR-LF, experiments were performed using procedures similar to the locomotion experiments. To demonstrate bidirectionality, the rotation direction of the actuator magnet was reversed mid-test. Obstacle traversal was demonstrated in a larger channel (ID: 25.4 mm) with an obstruction made from a clear adhesive gel-like putty (Clear Museum Gel, Quakehold!). Obstacle pushing was performed using a robot with a mass of 3 g and a cylindrical PLA obstacle with a mass of 15 g. Untethered drug delivery was demonstrated using food dye in a gelatin capsule that was placed within a perforated rigid compartment of an MR-LF. The demonstration showing catheter navigation was performed by injecting food dye through flexible tubing (TND80-010, Component Supply Co.) that was cast through the center of an MR-LF-S. Water tests were performed at 36° C. and involved robots moving within a thin, flexible plastic tubing (LDPE Poly Tubing, ID: 16 mm, thickness: 0.05 mm) suspended in an open configuration between two supports. For the demonstrations in FIGS. 9A to 10, the actuator magnet was located at a fixed position, and rotation occurred in the +z or −z direction. For the demonstrations in FIGS. 11A and 11B, the robot was turned by controlling the position and orientation of the actuator magnet in the x-z plane at yₐ=11 cm such that the rotation axis was approximately perpendicular to the robot's path.

1. A robot device, comprising:
a compartment configured to house one or more payload components; and
one or more feet joined to the compartment,
wherein at least one foot is joined to the compartment by way of a flexible connection, the at least one foot including a permanent magnet, and
wherein the at least one foot is configured to bend relative to the compartment when exposed to a magnetic field to thereby assist in locomotion of the robot device.

2. The robot device of claim 1, wherein the device comprises first and second feet, each of which includes a respective permanent magnet.

3. The robot device of claim 2, wherein the first and second feet are disposed on opposite sides of the compartment.

4. The robot device of claim 2, wherein both of the first and second feet are joined to the compartment by way of a respective first and second flexible connection.

5. The robot device of claim 4, wherein the first and second feet are cylindrically shaped, and wherein first and second flexible connections join at the center of the respective first and second feet.

6. The robot device of claim 4, wherein the compartment is a cylinder, wherein the first and second flexible connections join to end faces of the cylinder.

7. The robot device of claim 1, wherein the flexible connection has a length of about 1.25 mm to about 3.5 mm, or about 1.5 mm to about 3 mm, or about 1.75 mm to about 2.5 mm, or about 2 mm.

8. The robot device of claim 1, wherein the flexible connection has a diameter of about 2.5 mm to about 4.5 mm, or about 2.75 mm to about 4.25 mm, or about 2.75 mm to about 4 mm, or about 3 mm to about 3.75 mm, or about 3.6 mm.

9. The robot device of claim 1, wherein the robot device is sized to be ingestible by a human.

10. The robot device of claim 1, wherein the compartment has an internal volume of at least about 50 mm³, or at least about 100 mm³, or at least about 150 mm³, or at least about 200 mm³, or at least about 250 mm³, or about 300 mm³.

11. The robot device of claim 1, wherein the internal volume of the compartment comprises at least about 8% of the total volume of the robot device, or at least about 10% of the total volume of the robot device, or at least about 12% of the total volume of the robot device, or at least about 14% of the total volume of the robot device, or about 17% of the total volume of the robot device.

12. The robot device of claim 1, further comprising a payload within the compartment.

13. The robot device of claim 12, wherein the payload comprises a drug payload and/or one or more electronic components.

14. The robot device of claim 1, wherein the compartment is more rigid than the flexible connection.

15. The robot device of claim 1, wherein the outer diameter of the compartment is greater than the outer diameter of the flexible connection.

16. The robot device of claim 15, wherein the outer diameter of the at least one foot is greater than the outer diameter of the compartment.

17. The robot device of claim 1, further comprising a lumen extending through the robot device through which an intraluminal and/or diagnostic device may be disposed.

18. A robot system, comprising:
a robot device as in claim 1; and
a rotatable actuator magnet configured to drive rotation of the at least one foot of the robot device.

19. The robot system of claim 18, wherein adjustment of the actuator magnet's position, orientation, and rotation direction relative to the robot device enables the robot device to turn within a confined space and/or select a path at a bifurcation within a confined space.

20. A method of remotely delivering a payload to a target within a confined environment, the method comprising:
providing a robot device as in claim 1, wherein the robot device includes the payload within the compartment; and
actuating the robot device to cause locomotion of the robot device through the confined environment toward the target.

21. The method of claim 20, further comprising activating and/or releasing the payload upon reaching the target.

22. The method of claim 20, wherein the confined environment is a luminal space within human anatomy.
